SEQUENCE LISTING (16 sequences)

SEQ ID NO: 1 - protein, 28 aa, Homo sapiens
EGTFTSDVSSYLEGQAAKEFIAWLVKGR

SEQ ID NO: 2 - protein, 29 aa, Homo sapiens
EGTFTSDVSSYLEGQAAKEFIAWLVKGRG

SEQ ID NO: 3 - protein, 29 aa, Artificial
  synthetic
EGTFTSDVSSYLEGQAAKEFIAWLVKGRR

SEQ ID NO: 4 - protein, 28 aa, Artificial
  synthetic
EGTFTSDVSSYLEGQAAKEFIAWLVKGR

SEQ ID NO: 5 - protein, 29 aa, Artificial
  synthetic
EGTFTSDVSSYLEGQAAKEFIAWLVKGRG

SEQ ID NO: 6 - protein, 29 aa, Artificial
  synthetic
EGTFTSDVSSYLEGQAAKEFIAWLVKGRR

SEQ ID NO: 7 - protein, 28 aa, Artificial
  synthetic
EGTFTSDVSSYLEGQAAKEFIAWLVKGR

SEQ ID NO: 8 - protein, 29 aa, Artificial
  synthetic
EGTFTSDVSSYLEGQAAKEFIAWLVKGRG

SEQ ID NO: 9 - protein, 29 aa, Artificial
  synthetic
EGTFTSDVSSYLEGQAAKEFIAWLVKGRR

SEQ ID NO: 10 - protein, 28 aa, Artificial
  synthetic
EGTFTSDVSSYLEGQAAKEFIAWLVKGR

SEQ ID NO: 11 - protein, 29 aa, Artificial
  synthetic
EGTFTSDVSSYLEGQAAKEFIAWLVKGRR

SEQ ID NO: 12 - protein, 29 aa, Artificial
  synthetic
EGTFTSDVSSYLEGQAAKEFIAWLVKGRG

SEQ ID NO: 13 - protein, 30 aa, Artificial
  synthetic
EGTFTSDVSSYLEGQAAKEFIAWLVKGRGR

SEQ ID NO: 14 - protein, 30 aa, Artificial; identical to SEQ ID NO: 13
  synthetic

SEQ ID NO: 15 - protein, 30 aa, Artificial; identical to SEQ ID NO: 13
  synthetic

SEQ ID NO: 16 - protein, 30 aa, Artificial; identical to SEQ ID NO: 13
  synthetic

(19) United States
(12) Patent Application Publication
(10) Pub. No.: US 2008/0194483 A1
(43) Pub. Date: Aug. 14, 2008

(54) GLP-1 (9-36) methods and compositions

(76) Inventor: Michael A. Brownlee, New York, NY (US)

Correspondence Address: AMSTER, ROTHSTEIN & EBENSTEIN LLP, 90 PARK AVENUE, NEW YORK, NY, 10016, US

(21) Appl. No.: 12/008,362

(22) Filed: Jan. 10, 2008

Related U.S. Application Data
(63) Continuation-in-part of application No. 10/582,116, filed on Jun. 26, 2007.
(60) Provisional application No. 60/529,247, filed on Dec. 12, 2003.

(51) Int. Cl. A61K 38/22; C12N 5/06; A61P 3/10; A61P 9/10

(52) U.S. Cl. 514/12; 435/375

(57) ABSTRACT

Methods of inhibiting hyperglycemia-induced or free fatty acid-induced reactive oxygen formation in mammalian cells and mammals using the degradation product of glucagon-like peptide 1, GLP-1 (9-36) are provided. Various GLP-1 (9-36) compositions are also provided.

### CROSS-REFERENCE TO RELATED APPLICATIONS

This application is a continuation-in-part of U.S. patent application Ser. No. 10,582,116, filed on Jun. 26, 2007, which is a 35 U.S.C. §371 national stage of PCT International Patent Application No. PCT/US2004/040852, filed on Dec. 7, 2004, and claims the benefit of U.S. Provisional Application No. 60/529,247, filed on Dec. 12, 2003, the contents of which are incorporated by reference.

### FIELD OF THE INVENTION

The present invention generally relates to treatments for complications of diabetes and other disorders involving hyperglycemia. More specifically, the invention relates to treatments that reduce reactive oxygen formation induced by hyperglycemia or free fatty acids.

### BACKGROUND OF THE INVENTION

Various publications are referred to throughout this application. Citations for these references may be found at the end of the specification immediately preceding the claims. The disclosures of these publications are hereby incorporated by reference in their entireties into the subject application to more fully describe the art to which the subject application pertains.

Diabetes causes a variety of pathological changes in capillaries, arteries, and peripheral nerves. Diabetes-specific microvascular disease is the leading cause of blindness, renal failure, and nerve damage, and diabetes-associated atherosclerosis causes high rates of heart attack, stroke, and limb amputation. Seventy percent of all heart attack patients have either diabetes or impaired glucose tolerance.

Large prospective clinical studies in both type 1 and type 2 diabetic patients have shown that there is a strong relationship between the level of hyperglycemia and both onset and progression of diabetic microvascular complications in the retina, kidney, and peripheral nerve (DCCTRG, 1993; UKPDSG, 1998). Hyperglycemia also appears to have an important role in the pathogenesis of diabetic macrovascular disease (UKPDSG, 1998; Wei et al., 1998). Four major molecular mechanisms have been implicated in hyperglycemia-induced tissue damage: activation of protein kinase C (PKC) isoforms via de novo synthesis of the lipid second messenger diacylglycerol (DAG), increased hexosamine pathway flux, increased advanced glycation endproduct (AGE) formation, and increased polyol pathway flux. In aortic endothelial cells, hyperglycemia also activates the proinflammatory transcription factor NFκB and inactivates two important anti-atherogenic enzymes: prostacyclin synthase and endothelial nitric oxide synthase. Recently, it has been shown that all of these mechanisms reflect a single hyperglycemia-induced process: overproduction of superoxide (or reactive oxygen) by the mitochondrial electron transport chain (Brownlee, 2001; Nishikawa et al., 2000).

Glucagon-like peptide-1 (GLP-1) is synthesized in intestinal endocrine cells, in response to nutrient ingestion (Orskov et al., 1994), by differential processing of pro-glucagon into 2 principal major molecular forms—GLP-1 (7-36)amide and GLP-1 (7-37). The peptide was first identified following the cloning of cDNAs and genes for proglucagon in the early 1980s.

Initial studies of GLP-1 biological activity in the mid 1980s utilized the full length N-terminal extended forms of GLP-1 (1-37 and 1-36amide). These larger GLP-1 molecules were generally found to be devoid of biological activity. In 1987, 3 independent research groups demonstrated that removal of the first 6 amino acids resulted in a shorter version of the GLP-1 molecule with substantially enhanced biological activity.

The majority of circulating biologically active GLP-1 is found in the GLP-1 (7-36)amide form. The known major biological effects of GLP-1 (7-36) include stimulation of glucose-dependent insulin secretion and insulin biosynthesis, inhibition of glucagon secretion and gastric emptying, and inhibition of food intake (Drucker, 1998). The finding that GLP-1 lowers blood glucose in patients with diabetes, taken together with suggestions that GLP-1 may restore β cell sensitivity to exogenous secretagogues, suggests that augmenting GLP-1 signaling is a useful strategy for treatment of diabetic patients. Mounting evidence strongly suggests that GLP-1 signaling regulates islet proliferation and islet neogenesis (Buteau et al., 1999).

GLP-1 is rapidly inactivated to its degradation products GLP-1 (9-36 amide) and GLP-1 (9-37) by the enzyme dipeptidyl peptidase IV (DPP IV). DPP IV-mediated inactivation is a critical control mechanism for regulating the biological activity of GLP-1 in vivo in both rodents and humans (Mentlelin et al., 1993; Kieffer et al., 1995; Deacon et al., 1995a and b). Several studies have also implicated a role for neutral endopeptidase 24.11 in the endoproteolysis of GLP-1 (Hupe-Sodmann et al., 1995; Hupe-Sodmann et al., 1997).

DPP IV inhibitors, and more-slowly degrading analogs of GLP-1 (7-36) are currently in use for therapeutic purposes. GLP-1 analogues that are resistant to DPP IV cleavage are more potent in vivo. An example of a naturally occurring DPP IV-resistant GLP-1 analogue is lizard exendin-4 (Edwards et al., 2001).

There have been a few reports indicating that GLP-1 (9-36) has some biological activity. Deacon et al., 2002, provides data indicating that GLP-1 (9-36) reduces total blood glucose somewhat 10-20 minutes after glucose infuision. This small reduction in blood glucose would not be expected to affect hyperglycemia-induced reactive oxygen formation, however. Additionally, Wettergren et al., 1998, found no effect from GLP-1 (9-36) on atrial motility. Neither Deacon et al. nor Wettergren et al. indicate that GLP-1 (9-36) is capable of inhibiting hyperglycemia-induced or fatty acid-induced reactive oxygen formation and its consequences.

Three patent publications, WO 03/061362, WO 02/085406 and US 2003/0073626, have claims to therapeutic treatments using GLP-1 (9-36). However, those publications do not provide an enabling disclosure of any GLP-1 (9-36) activity.

There is thus a need for new treatments that reduce or eliminate hyperglycemia-induced reactive oxygen species, in order to reduce complications of diabetes. There is also a need to determine whether GLP-1 (9-36) has any clinically significant activity. The present invention addresses both of these needs.

### SUMMARY OF THE INVENTION

Accordingly, the inventor has discovered that GLP-1 (9-36amide) and GLP-1 (9-37) inhibit hyperglycemia-induced reactive oxygen formation in mammalian cells. Based on this discovery, methods and compositions are provided that are useful for inhibiting various disorders caused by reactive oxygen.

Thus, in some embodiments, the invention is directed to methods of inhibiting hyperglycemia-induced or free fatty acid-induced reactive oxygen formation in a mammalian nerve cell, renal mesangial cell, pancreatic P cell, adipocyte, cardiac myocyte or, preferably an endothelial cell or hepatocyte. The methods comprise treating the cell with a pharmaceutically acceptable composition comprising GLP-1 (9-36) sufficient to inhibit the hyperglycemia-induced or free fatty acid-induced reactive oxygen formation in the cell.

In other embodiments, the invention is directed to methods of inhibiting the development of disease due to diabetes, impaired glucose tolerance, stress hyperglycemia, metabolic syndrome, and/or insulin resistance in a mammal, or conditions resulting therefrom. The methods comprise treating the mammal with a pharmaceutically acceptable composition comprising GLP-1 (9-36) sufficient to inhibit hyperglycemia-induced or free fatty acid-induced reactive oxygen formation in the mammal.

The invention is also directed to methods of reducing hyperglycemia-induced or free fatty acid-induced inactivation of prostacyclin synthase in a mammal. The methods comprise treating the mammal with GLP-1 (9-36) sufficient to inhibit the hyperglycemia-induced or free fatty acid-induced reactive oxygen formation in the mammal.

The invention is further directed to methods of inhibiting hyperglycemia-induced or free fatty acid-induced decrease in endothelial nitric oxide synthase (eNOS) activity in an endothelial cell. The methods comprise treating the mammal with GLP-1 (9-36) sufficient to inhibit the hyperglycemia-induced or free fatty acid-induced decrease in eNOS activity in the cell.

In additional embodiments, the invention is directed to isolated and purified GLP-1 (9-36) consisting essentially of a sequence selected from the group consisting of SEQ ID NOs:3, 4, 5, 6, 7, 8, 9, 10, 11, 12, 13, 14, 15 and 16. Compositions comprising GLP-1 (9-36) forms in a pharmaceutically acceptable excipient, are also provided. The compositions can include GLP-1 (9-36) modified with a fatty acid to create a slow-release form. The compositions can have an extra basic amino acid added to decrease the solubility at physiologic pH and so create a slow-release form.

### BRIEF DESCRIPTION OF THE DRAWINGS

FIG. 1 is a graph of experimental results establishing that GLP-1 (9-36amide) prevents hyperglycemia-induced reactive oxygen production in vascular endothelial cells.

FIG. 2 is a graph of experimental results establishing that GLP-1 (9-36amide) prevents hyperglycemia-induced decreases in endothelial nitric oxide synthase activity in vascular endothelial cells.

FIG. 3 is a graph of experimental results establishing that GLP-1 (9-36amide) prevents diabetes-induced inactivation/inhibition of prostacyclin synthase in aortas from treated diabetic mice.

FIG. 4 is a graph of experimental results establishing that GLP-1 (9-36amide) prevents hyperglycemia-induced reactive oxygen production in hepatocytes.

### DETAILED DESCRIPTION OF THE INVENTION

The present invention is based on the discovery that GLP-1 (9-36amide) and GLP-1 (9-37) inhibit hyperglycemia-induced reactive oxygen formation in mammalian cells. This discovery leads to the use of GLP-1 (9-36) and similar compounds for the treatment of complications caused by reactive oxygen species and reactive nitrogen species.

Thus, in some embodiments, the invention is directed to methods of inhibiting hyperglycemia-induced or free fatty acid-induced reactive oxygen formation in a mammalian cell. The methods comprise treating the cell with a pharmaceutically acceptable composition comprising GLP-1 (9-36) sufficient to inhibit the hyperglycemia-induced or free fatty acid-induced reactive oxygen formation in the cell. The cell is preferably part of a living mammal.

In preferred embodiments, the reactive oxygen formation is hyperglycemia induced, however, since free fatty acids are known to induce reactive oxygen (See, e.g., U.S. Provisional Patent App. No. 60/474,520 and references cited therein), that induction would also be expected to be affected by GLP-1 (9-36).

The cell is any cell that is capable of producing reactive oxygen in response to hyperglycemia or free fatty acids. The cell is preferably a cell that is affected by reactive oxygen to cause complications associated with hyperglycemia or free fatty acids, for example a nerve cell, a renal mesangial cell, a pancreatic β cell, an adipocyte, a cardiac myocyte, an endothelial cell or a hepatocyte.

In some preferred embodiments, the cell is an endothelial cell, preferably a vascular endothelial cell. The endothelial cell is preferably in a mammal (most preferably a human) that has or is at risk for having diabetes, impaired glucose intolerance, stress hyperglycemia, metabolic syndrome, and/or insulin resistance. The methods would also be useful for a critically ill mammal, since hyperglycemic mechanisms are risk factors in critically ill patients, even when they were not diabetic (Van den Berghe et al., 2001). Complications from chronic ischemia would also be usefully treated with any of the various GLP-1 (9-36) forms since hyperglycemia-induced reactive oxygen species (ROS) impair the normal reparative response to chronic ischemia.

In other preferred embodiments, the cell is a hepatocyte, preferably in a living mammal that has or is at risk for ischemia/reperfusion injury, endotoxin injury, alcoholic liver disease or non-alcoholic steatohepatitis (NASH), which is associated with diabetes and is becoming the primary cause of cirrhosis. See also Example 4, showing that treatment of hepatocytes with GLP-1 (9-36amide) also beneficially reduces hyperglycemia-induced reactive oxygen formation.

In additional preferred embodiments, the cell is a β cell, preferably in a living mammal that has or is at risk for impaired glucose-stimulated insulin secretion.

In these methods, the GLP-1 (9-36) preferably has the sequence of SEQ ID NO:1. However, the term “GLP-1 (9-36)” is not limited to SEQ ID NO:1, but could also include any of SEQ ID NO:2-16, since each of those sequences are expected to be useful for reducing reactive oxygen formation induced by hyperglycemia or free fatty acids. Specifically, SEQ ID NO:2 is naturally occurring GLP-1 (9-37), i.e., GLP-1 (9-36) along with the 37^{th} amino acid of GLP-1, Gly.

GLP-1 (9-36) can also usefully comprise an additional arginine (GLP-1 (9-36+arg37)) (SEQ ID NO:3) to raise the isoelectric point, giving the peptide reduced solubility and slower degradation at physiologic pH, similar to insulin glargine, a long-acting insulin derivative. Other amino acid changes that raise the isoelectric point towards physiological pH would also have slower degradation.

Acylation of the ε-amino group of Lys B29 in insulin with myristoylic acid promotes reversible binding of insulin to albumin, thereby delaying absorption from the subcutaneous injection site. With GLP-1 (9-36), similar acylation could be accomplished at Lys 26, Lys 34, and/or at the amino terminus in combination with any of the previously described GLP-1 (9-36) (SEQ ID NO:4-16). Such peptides could be, e.g., injected subcutaneously, or administered by inhalation of modified peptides encapsulated in a biodegradable polymer as described in Edwards, D., et al., 1997; VanBever, R. et al., 1999; and Hrkach, 2000.

Additionally, each of the sequences SEQ ID NO:1-16 could also be an amide, since the amide of GLP-1 (7-36) is the naturally occurring active form of this peptide.

The GLP-1 (9-36) forms described above can be made by any known method, e.g., enzymatic digestion of a larger form, for example using DPP IV, expression of the peptide using an expression vector comprising a nucleotide sequence that encodes the GLP-1 (9-36), or, preferably, by chemical synthesis.

The GLP-1 (9-36) can also be a peptidomimetic, as are known in the art.

In some embodiments, it may also be useful to evaluate the effectiveness of these methods by known methods, for example by directly measuring reactive oxygen in the cell.

Another method of evaluating the effectiveness of these methods is by measuring prostacyclin synthase activity in the endothelial cell and/or in serum or plasma from patients, since prostacyclin synthase is very sensitive to inactivation by reactive oxygen (see, e.g., Example 3). The prostacyclin synthase can be measured by any known method. A preferred method is measuring the formation of 6-keto-PGF_{1α} (Example 3).

This invention could be used in both prophylactic and therapeutic regimens. For prophylactic use, patients with Type I or Type II diabetes, impaired glucose tolerance, the metabolic syndrome, or stress hyperglycemia, would continuously take the pharmaceutical GLP-1 (9-36) composition along with their usual medical regimen to diminish complications due to the increased formation of reactive oxygen species. For therapeutic use, these inhibitors would be administered at the time of the ischemic event to decrease subsequent morbidity and mortality.

When the endothelial cell is in a living mammal, the GLP-1 (9-36) composition can be formulated without undue experimentation for administration to the mammal, including humans, as appropriate for the particular application. Additionally, proper dosages of the GLP-1 (9-36) compositions can be determined without undue experimentation using standard dose-response protocols. Preferred methods of administration include administration by intravenous, intramuscular or subcutaneous injection and by subcutaneous infusion pump. However, the invention is not narrowly limited to any particular methods of administration.

In many of the above-described methods, the GLP-1 (9-36) is formulated in a slow release composition by standard methods, for example a microcrystalline composition.

The GLP-1 (9-36) compositions of the present invention can easily be administered parenterally such as for example, by intravenous, intramuscular, intrathecal or subcutaneous injection, or by subcutaneous infusion pump. Parenteral administration can be accomplished by incorporating the compositions of the present invention into a solution or suspension. Such solutions or suspensions may also include sterile diluents such as water for injection, saline solution, fixed oils, polyethylene glycols, glycerine, propylene glycol or other synthetic solvents. Parenteral formulations may also include antibacterial agents such as for example, benzyl alcohol or methyl parabens, antioxidants such as for example, ascorbic acid or sodium bisulfite and chelating agents such as EDTA. Buffers such as acetates, citrates or phosphates and agents for the adjustment of tonicity such as sodium chloride or dextrose may also be added. The parenteral preparation can be enclosed in ampules, disposable syringes or multiple dose vials made of glass or plastic.

Rectal administration includes administering the GLP-1 (9-36) pharmaceutical compositions into the rectum or large intestine. This can be accomplished using suppositories or enemas. Suppository formulations can easily be made by methods known in the art. For example, suppository formulations can be prepared by heating glycerin to about 120° C., dissolving the composition in the glycerin, mixing the heated glycerin after which purified water may be added, and pouring the hot mixture into a suppository mold.

Transdermal administration includes percutaneous absorption of the composition through the skin. Transdermal formulations include patches (such as the well-known nicotine patch), ointments, creams, gels, salves and the like.

The present invention includes nasally administering to the mammal a therapeutically effective amount of the composition. As used herein, nasally administering or nasal administration includes administering the composition to the mucous membranes of the nasal passage or nasal cavity of the patient. As used herein, pharmaceutical compositions for nasal administration of a composition include therapeutically effective amounts of the composition prepared by well-known methods to be administered, for example, as a nasal spray, nasal drop, suspension, gel, ointment, cream or powder. Administration of the composition may also take place using a nasal tampon or nasal sponge.

The GLP-1 (9-36) compositions can also be administered to the mammal with at least one other treatment for inhibiting the effects of diabetes, impaired glucose tolerance, stress hyperglycemia, metabolic syndrome, and/or insulin resistance. One example of such treatments is administration of insulin. Various other treatments are discussed in U.S. Provisional Patent App. No. 60/474,520, incorporated herein by reference.

Another example of a treatment that can be administered with the GLP-1 (9-36) composition is a treatment that inhibits poly(ADP-ribose) polymerase (PARP) activity or accumulation in the mammal. It is known that hyperglycemia-induced mitochondrial superoxide overproduction activates poly (ADP-ribose) polymerase (PARP). PARP activation, in turn, inhibits glyceraldehyde-3-phosphate dehydrogenase (GAPDH) activity which activates at least three of the major pathways of hyperglycemic damage in endothelial cells. Inhibiting PARP activity thus inhibits the development of complications of diabetes. See U.S. Provisional Patent App. No. 60/474,520. Such treatments include administration of a PARP inhibitor. Nonlimiting examples of PARP inhibitors include PJ34, 3-aminobenzamide, 4-amino-1,8-naphthalimide, 6(5H)-phenanthridinone, benzamide, INO-1001, and NU1025. PARP activity can also be inhibited by administering to the mammal a nucleic acid or mimetic that specifically inhibits transcription or translation of the PARP gene. Examples of such nucleic acids or mimetics include an antisense complementary to mRNA of the PARP gene, a ribozyme capable of specifically cleaving the mRNA of the PARP gene, and an RNAi molecule complementary to a portion of the PARP gene. PARP activity can also be inhibited by administration of a compound that specifically binds to the PARP, such as an antibody or an aptamer.

An additional example of a treatment that can be administered with the GLP-1 (9-36) composition is a treatment that activates transketolase in the mammal. See U.S. Provisional Patent App. No. 60/474,520. A preferred method of activating transketolase is by administering a lipid-soluble thiamine derivative to the mammal. Examples of such lipid-soluble thiamine derivatives are benfotiamine, thiamine propyl disulfide, and thiamine tetrahydrofurfuryl disulfide.

Another treatment that can be administered with the GLP-1 (9-36) composition is a treatment that further reduces superoxide in the mammal. Such treatments include administration of an α-lipoic acid, a superoxide dismutase mimetic or a catalase mimetic. Examples of superoxide dismutase mimetics and catalase mimetics include MnTBAP, ZnTBAP, SC-55858, EUK-134, M40403, AEOL 10112, AEOL 10113 and AEOL 10150.

A further treatment that can be administered with the GLP-1 (9-36) composition is a treatment that inhibits excessive release of free fatty acids in the mammal. See U.S. Provisional Patent App. No. 60/474,520. Examples of treatments that inhibit excessive release of free fatty acids are the administration of compounds such as a thiazolidinedione, nicotinic acid, adiponectin and acipimox.

In other embodiments, the invention is directed to methods of inhibiting the development of disease due to diabetes, impaired glucose tolerance, stress hyperglycemia, metabolic syndrome, and/or insulin resistance in a mammal, or conditions resulting therefrom. The methods comprise treating the mammal with a pharmaceutically acceptable composition comprising GLP-1 (9-36) sufficient to inhibit hyperglycemia-induced or free fatty acid-induced reactive oxygen formation in the mammal. These methods would be expected to be effective in any mammal, including humans.

Nonlimiting examples of diseases that are inhibited by these methods include atherosclerotic, microvascular, or neurologic disease, such as coronary disease, myocardial infarction, atherosclerotic peripheral vascular disease, cerebrovascular disease, stroke, retinopathy, renal disease, neuropathy, and cardiomyopathy.

As with the previously described methods, the GLP-1 (9-36) composition of these methods can also be administered with at least one other treatment for inhibiting the effects of diabetes, impaired glucose tolerance, stress hyperglycemia, metabolic syndrome, and/or insulin resistance. Such methods have been described above, and in U.S. Provisional Patent App. No. 60/474,520.

In normal animals and people, the endothelial cell enzyme prostacyclin synthase prevents excessive platelet aggregation, and has a variety of other anti-atherogenic actions. Prostacyclin synthase can also protect against development of hypoxic pulmonary hypertension (Geraci et al., 1999). In addition, loss of prostacyclin synthase shifts arachadonic acid metabolism toward increased thromboxaneA2, lipoxygenase, etc., which have further adverse effects on vessel. The inventor has discovered that treatment with GLP-1 (9-36) protects prostacyclin synthase from hyperglycemia-induced reactive oxygen formation, and is thus a useful treatment for maintaining active prostacyclin synthase. See Example 3.

Thus, in additional embodiments, the invention is directed to methods of reducing hyperglycemia-induced or free fatty acid-induced inactivation of prostacyclin synthase in a mammal. The methods comprise treating the mammal with GLP-1 (9-36) sufficient to inhibit the hyperglycemia-induced or free fatty acid-induced reactive oxygen formation in the mammal.

In some preferred embodiments, the mammal treated in these methods has or is at risk for hypoxic pulmonary hypertension. In other preferred embodiments, the mammal is at risk for undergoing an acute thrombotic event such as a stroke or a heart attack.

As shown in Example 2, treatment with GLP-1 (9-36) also beneficially reduces hyperglycemia- or free fatty acid-induced decrease in nitric oxide synthase (eNOS). Normal endothelial production of nitric oxide plays an important role in preventing vascular disease. In addition to its function as an endogenous vasodilator, nitric oxide released from endothelial cells is a potent inhibitor of platelet aggregation and adhesion to the vascular wall. Endothelial NO also controls the expression of genes involved in atherogenesis. It decreases expression of the chemoattractant protein MCP-1, and of surface adhesion molecules such as CD11/CD18, P-selectin, vascular cell adhesion molecule-1 (VCAM-1) and intercellular adhesion molecule-1 (ICAM-1). Endothelial cell nitric oxide also reduces vascular permeability, and decreases the rate of oxidation of low density lipoprotein to its pro-atherogenic form. Finally, endothelial cell nitric oxide inhibits proliferation of vascular smooth muscle cells. Endothelium-dependent vasodilation is impaired in both microcirculation and macrocirculation during acute hyperglycemia in normal subjects as well as in diabetic patients, suggesting that nitric oxide synthase activity may be chronically impaired in diabetic patients.

Thus, the present invention is also directed to methods of inhibiting hyperglycemia-induced or free fatty acid-induced decrease in endothelial nitric oxide synthase (eNOS) activity in an endothelial cell. The methods comprise treating the mammal with GLP-1 (9-36) sufficient to inhibit the hyperglycemia-induced or free fatty acid-induced decrease in eNOS activity in the cell. As with the analogous methods described above relating to reactive oxygen, the endothelial cell can be part of the vascular tissue of a living mammal, preferably a human. In preferred embodiments, the living mammal has or is at risk for having diabetes, impaired glucose intolerance, stress hyperglycemia, metabolic syndrome, and/or insulin resistance.

Also as with the methods described above relating to reactive oxygen, any GLP-1 (9-36) form having the sequence of any of SEQ ID NO:3-16 can be utilized with these methods to provide a longer lasting peptide composition.

The invention is also directed to novel forms of GLP-1 (9-36), for example the sequences of SEQ ID NOs:3, 4, 5, 6, 7, 8, 9, 10, 11, 12, 13, 14, 15, and 16. Preferably, the novel GLP-1 (9-36) is isolated and purified. Where these novel forms of GLP-1 (9-36) are used therapeutically, they are usefully formulated in a pharmaceutically acceptable excipient, and are also preferably an amide.

Examples of these novel forms of GLP-1 (9-36) include a GLP-1 (9-36) that further comprises an additional Arg at the carboxy terminus; a GLP-1 (9-36) that comprises at least one acetylated lysine or N-terminal amino group, for example where the acetyl group is a myristoyl group.

Preferred embodiments of the invention are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims which follow the examples.

### EXPERIMENTAL DETAILS

### EXAMPLE 1

### GLP-1 (9-36) Prevents Hyperglycemia-Induced Reactive Oxygen Production in Vascular Endothelial Cells

Cultured vascular endothelial cells were treated with GLP-1 (9-36) to determine the effect of GLP-1 (9-36) on hyperglycemia-induced reactive oxygen production by those cells.

### Materials and Methods

Cell culture conditions. For reactive oxygen species (RO) measurement, bovine aortic endothelial cells (BAECs, passage 4-10) were plated in 96 well plates at 100,000 cells/well in Eagle's MEM containing 10% FBS, essential and nonessential amino acids, and antibiotics. Cells were incubated with either 5 mM glucose, 30 mM glucose, 30 mM glucose plus 10 nM GLP-1 (7-36), 30 mM glucose plus 10 nM GLP-1 (7-36), 30 mM glucose plus 10 nM GLP-1 (7-36) plus 10 μM pyrrolidide (a DPP IV inhibitor), 30 mM glucose plus 10 nM GLP-1 (7-36) plus 10 μM pyrrolidide and 100 μM phosphoramidon (a neutral endopeptidase 24.11 inhibitor), and 30 mM glucose plus GLP-1 (9-36) plus 10 nM exendin 9-39, a blocker of the GLP-1 (7-36) receptor. The pyrrolidide, phosphoramidon, and exendin 9-39 were each added to the cells four hours before the addition of the peptides. The ROS measurements were performed 24 hrs later.

Intracellular reactive oxygen species measurements. The intracellular formation of reactive oxygen species was detected using the fluorescent probe CM-H₂DCFDA (Molecular Probes). Cells (1×10⁵ ml⁻¹) were loaded with 10 μM CM-H₂DCFDDA, incubated for 45 min at 37° C., and analysed in an HTS 7000 Bio Assay Fluorescent Plate Reader (Perkin Elmer) using the HTSoft program. ROS production was determined from an H₂O₂ standard curve (10-200 nmol ml⁻¹).

### Results and Discussion

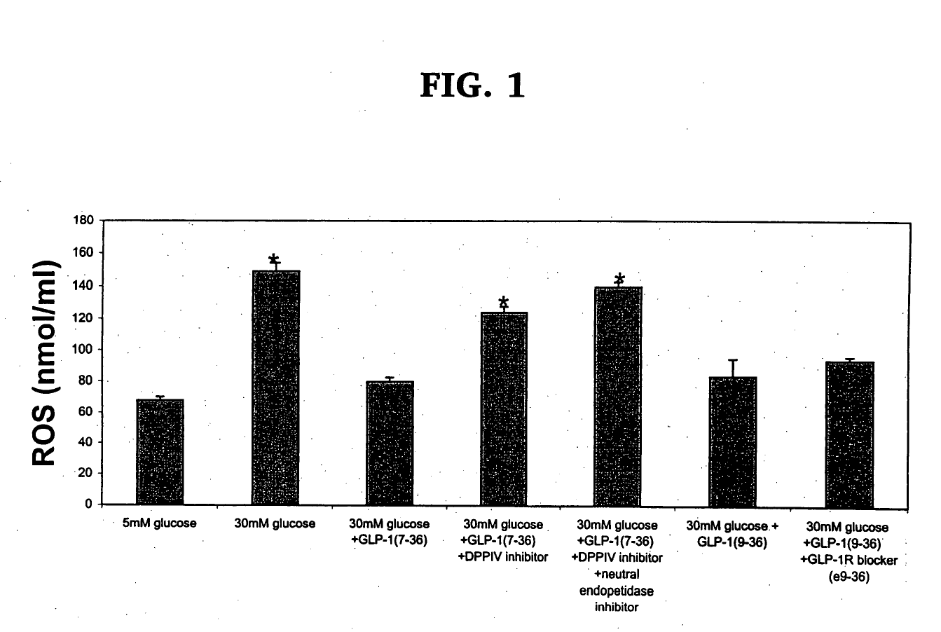

As shown in FIG. 1, GLP-1 (9-36) inhibited production of ROS in vascular endothelial cells in culture. Diabetic levels of hyperglycemia or free fatty acids cause increased ROS production in these cells (FIG. 1, bar 2). Adding GLP-1 (7-36) completely prevents this damaging effect (FIG. 1, bar 3). However, when GLP-1 degradation is blocked by inhibitors of enzymes that cleave GLP-1 (FIG. 1, bars 4 and 5), the intact GLP-1 (7-36) has no effect on hyperglycemia-induced ROS.

In contrast, addition of the “inactive” GLP-1 degradation product (FIG. 1, bar 6), completely inhibits hyperglycemia-induced overproduction of ROS. Furthermore, blockade of the GLP-1 receptor with e9-39 has no effect on this property, strongly suggesting that the effect is mediated through a different, undiscovered receptor.

Thus, the degradation product of GLP-1, previously thought to be biologically inactive, has a profound effect on vascular endothelial cells—it prevents completely hyperglycemia-induced overproduction of superoxide (FIG. 1).

### EXAMPLE 2

### GLP-1 (9-36) Prevents Hyperglycemia- and Fatty Acid-Induced Decreases in Endothelial Nitric Oxide Synthase (eNOS) Activity in Vascular Endothelial Cells

Cultured vascular endothelial cells were treated with GLP-1 (9-36) to determine the effect of GLP-1 (9-36) on hyperglycemia-induced decreases in eNOS activity in those cells.

### Materials and Methods

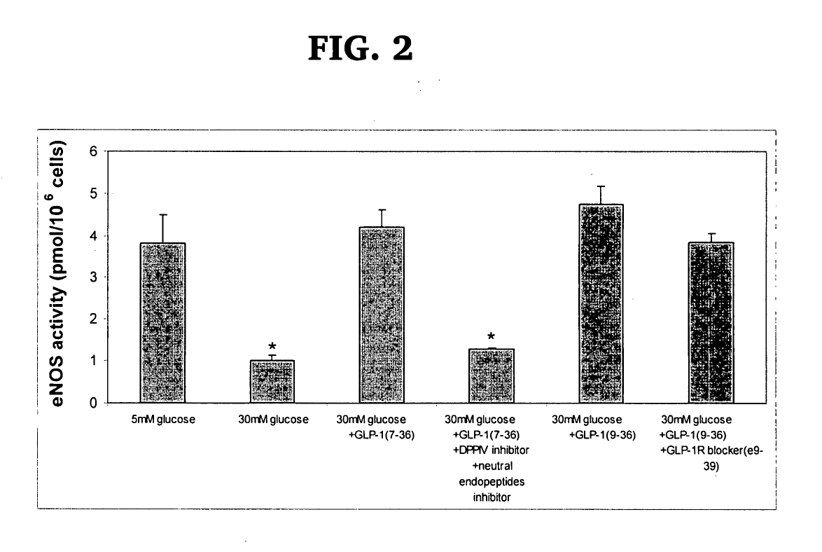

Cell-culture conditions. For measurement of endothelial nitric oxide activity (eNOS), bovine aortic endothelial cells (BAECs, passage 4-10) were plated in 24 well plates at 200,000 cells /well in Eagle's MEM containing 10% FBS, essential and nonessential amino acids, and antibiotics. Cells were incubated with either 5 mM glucose, 30 mM glucose, 30 mM glucose plus 10 nM GLP-1 (7-36), 30 mM glucose plus 10 nM GLP-1 (7-36) plus 10 μM pyrrolidide (a DPP IV inhibitor) (not shown in FIG. 2), 30 mM glucose plus 10 nM GLP-1 (7-36) plus 10 μM pyrrolidide and 100 μM phosphoramidon (a neutral endopeptidase 24.11 inhibitor), 30 mM glucose+GLP-1 (9-36), and 30 mM glucose plus GLP-1 (9-36) plus 10 nM exendin 9-39, a blocker of the GLP-1 (7-36) receptor. The pyrrolidide, phosphoramidon, and exendin 9-39 were each added to the cells four hours before the addition of the peptides. eNOS activity measurements were performed 48 hrs later.

Measurement of eNOS activity. eNOS activity in cells was determined by first incubating cells in L-arginine-deficient, serum-free MEM media for 6 hours. This media was then replaced with PBS buffer containing 120 mM NaCl, 4.2 mM KCl, 2.5 mM CaCl₂, 1.3 mM MgSO₄, 1.2 mM Na₂HPO₄, 0.37 mM KH₂PO₄, 10 mM HEPES, and 7.5 mM glucose (500 μl/well); cells were then incubated for 15 minutes at 37° C. The eNOS activity assay was initiated by incubating cells with PBS buffer (400 μl/well) containing 1.5 Ci/ml [³H]L-arginine for 15 minutes. The reaction was stopped by adding 1 N ice-cold TCA (500 μl/well). Cytosol preparations were transferred to ice-cold silanized glass tubes and extracted three times with water-saturated ether. The samples were neutralized with 1.5 ml of 25 mM HEPES (pH 8.0) and applied to Dowex AG50WX8 columns (Tris form) (Sigma Chemical Co., St. Louis, Mo., USA). Columns were eluted with 1 ml of 40 mM HEPES buffer (pH 5.5) containing 2 mM EDTA and 2 mM EGTA. The eluate was collected in glass scintillation vials for [³H]L-citrulline quantitation by liquid scintillation spectroscopy.

### Results and Discussion

The results are summarized in FIG. 2. Diabetic levels of hyperglycemia cause decreased eNOS activity in these cells (FIG. 2, bar 2). Adding GLP-1 (9-36) completely prevents this damaging effect (FIG. 2, bar 3). However, when GLP-1 (7-36) degradation is blocked by enzyme inhibitors (FIG. 2, bar 4), the intact GLP-1 (7-36) has no effect on hyperglycemia-induced eNOS.

In contrast, addition of GLP-1 (9-36) (FIG. 2, bar 5), completely inhibits hyperglycemia-induced overproduction of ROS. Furthermore, blockade of the GLP-1 receptor with e9-39 (FIG. 2, bar 6) has no effect on this property, providing further evidence that the effect is mediated through a different, undiscovered receptor.

These results precisely mirrored the results with ROS discussed in Example 1, indicating a common mechanism.

### EXAMPLE 3

### GLP-1 (9-36) Prevents Diabetes-Induced Inactivation/Inhibition of Prostacyclin Synthase in Diabetic Mouse Aortas

In vivo studies were conducted to determine whether GLP-1 (9-36) has a physiologically relevant in vivo effect on prostacyclin synthase, which is strongly affected by reactive oxygen.

### Materials and Methods

Animal studies. Male C57B16 mice (6-8 weeks old) were made diabetic by daily injections of 50 mg/kg streptozotocin in 0.05 M NaCitrate pH 4.5 after an eight hour fast, for five consecutive days. Two weeks after the initial injection the blood glucose was determined and the diabetic mice were randomized into two groups with equal mean blood glucose levels. Alzet micro-osmotic pumps were inserted into 10 diabetic mice. The pump was filled with GLP-1 (9-36) peptide at a concentration of 10 μg/100 μl. Seven days later 10 untreated diabetic mice, 10 treated diabetic mice, and 10 non-diabetic control mice were sacrificed. Blood glucose was determined at time of sacrifice. The aorta was removed from the abdominal bifurcation to the aortic arch, and prostacyclin activity was determined by measurement of its stable product 6-keto-PGF_{1α}.

Measurement of 6-keto-PGF_{1α}. 6-keto-PGF_{1α} is a stable product which is produced by the non-enzymatic hydration of PGI₂. A competitive immunoassay method (Correlate-EIA) was used for the quantitative determination of 6-keto-PGF_{1α}. The samples were prepared from dissected mouse aortas. The aorta was dissected from the abdominal bifurcation to the aortic arch. Briefly, the aorta was washed with PBS and incubated at 37° C. for 3 hours in 400 μl incubation buffer which contained 20 mM Tris-HCl buffer (pH 7.5) and 15 μl arachidonic acid. 100 μl of sample was used to measure the 6-keto-PGF₁ₐ concentration according to the manufacturer's instructions (Assay Design Inc.). The data are expressed per aorta.

### Results and Discussion

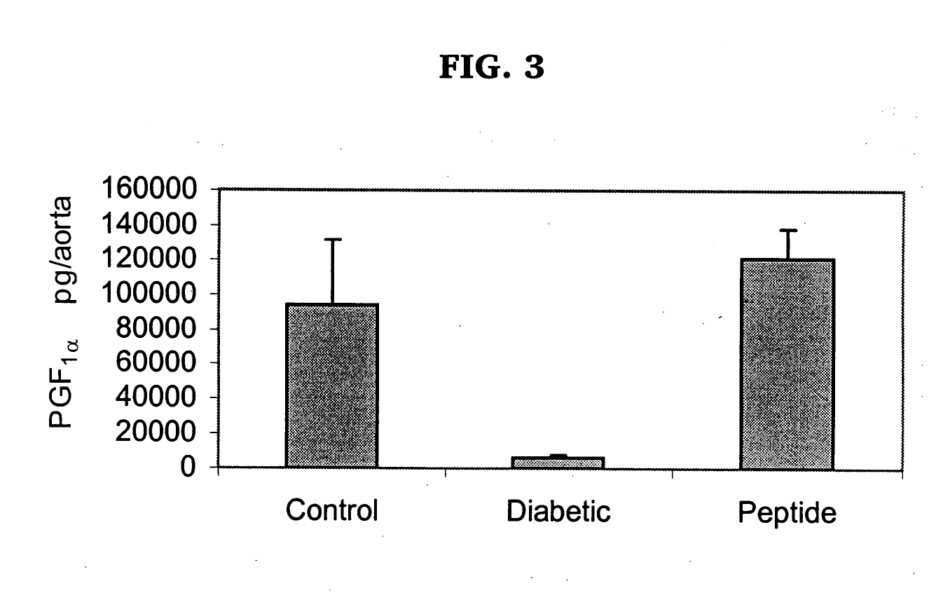

The results are summarized in FIG. 3. GLP-1 (9-36) (“Peptide”) completely eliminated the diabetes-induced inactivation of prostacyclin synthase. This shows that in vivo administration of GLP-1 (9-36) has a significant effect on diabetes-induced reactive oxygen formation and physiological systems affected by reactive oxygen.

### EXAMPLE 4

### GLP-1 (9-36) Prevents Hyperglycemia-Induced Reactive Oxygen Production in Hepatocytes

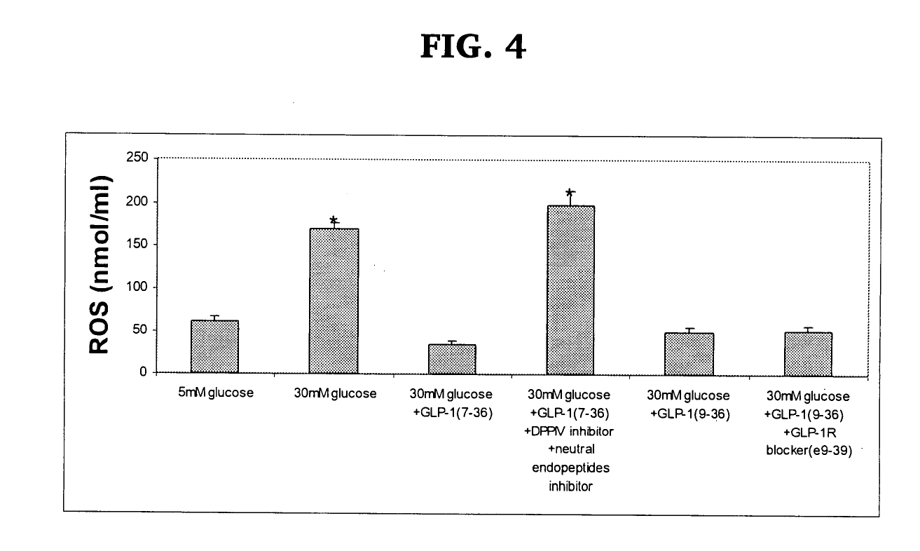

An experiment similar to that described in Example 1 was performed, using hepatocytes rather than endothelial cells. As shown in FIG. 4, GLP-1 (9-36) inhibited hyperglycemia-induced reactive oxygen species (ROS) formation in hepatocytes in a similar manner as with endothelial cells.

### EXAMPLE 5

### GLP-1 (9-36) Prevents Nutrient-Induced Endothelial Dysfunction

Insulin resistance and diabetes both cause nutrient-induced endothelial cell dysfunction by increasing mitochondrial superoxide production. In aortic endothelial cells, GLP-1(9-36) prevented both glucose- and fatty acid-induced ROS, and ROS-dependent inactivation of two important antiatherogenic enzymes. GLP-1(9-36) also normalized these parameters in diabetic GLP-1 receptor^{-/-} mice. Thus, continuous delivery of concentrations of the inactive metabolites of GLP-1 several-fold higher than what occurs in vivo prevent nutrient-induced elevation of ROS in human aortic endothelial cells, an effect which is independent of the classic GLP-1 receptor. As anti-diabetic therapy with dipeptidyl peptidase-4 (DPP-4) inhibitors prevents the formation of GLP-1(9-36), these findings have implications for the treatment of type 2 diabetes.

### Material and Methods

Materials. Human aortic endothelial cells (HAEC) were obtained from Cascade Biologics (Portland, Oreg.). EGM2 Media plus growth factor additives were obtained from Cambrex Bio Science, (Walkerville, Md.). Oleic acid, pure fatty acid free-albumin, cyclohexamide, DPP-4 inhibitor (valine pyrrolidide), NEP 24.11 inhibitor (phosphoramindon) and GLP-1R antagonist exendin (9-39 amide) were obtained from Sigma-Aldrich (St Louis, Mo.). CM-H2DCFDA was obtained from Invitrogen (Carlsbad, Calif.). 6-keto-PGF-1 α kits were obtained from Assay Designs (Ann Arbor, Mich., USA). Protein A agarose was from Roche (Nutley, N.J.). [³H]L-arginine was from GE Health Care Life Sciences (Piscataway, N.J.). eNOS antibody (#Sc-654) and phosphotyrosine antibody were from Santa Cruz Biotechnology Inc (Santa Cruz, Calif.). Alzet pumps were obtained from Durect Corporation (Cupertino, Calif.). GLP-1 (9-36)^{amide} was synthesized and purified by HPLC at Bachem (King of Prussia, Pa.). Anti-PGI2 (160640) was from Cayman (Ann Arbor Mich.).

Cell Culture conditions. Confluent HAECs (passage 1-6) were maintained in EGM2 media containing 0.4% FBS plus growth factor additives. Cells were incubated for varying times with either 5 mM glucose, 12 mM glucose, or 5 mM glucose plus 800 μM oleic acid and 1 mM albumin. In other experiments, cells were incubated with 5 mM glucose, 12 mM glucose, or 5 mM glucose plus 800 μM oleic acid and 1 mM albumin, alone, or with either 100 pM GLP-1 (7-36 NH₂), GLP-1 (7-36 NH₂)+ the DPPIV inhibitor H-lys(4-nito-Z)-pyrrolidide and the neutral endopeptidase inhibitor phosphoramidon, GLP-1 (9-36 NH₂), GLP-1 (9-36 NH₂) plus the GLP-1 receptor blocker exendin (9-36), and with GLP-1 (9-36 NH₂) with 100× GLP-1 (7-36 NH₂) and both DPPIV and NEP inhibitors. Cells were pre incubated for four hrs in cycloheximide (5 ug/ml). In experiments using clotrimazole cells were preincubated for 2.5 hrs in serum free media.

Reactive oxygen species quantification. Cells were plated in 96-well cell culture plates. Intracellular reactive oxygen species were detected using the fluorescent probe CM-H2DCFDA (Molecular Probes). Cells were loaded with 10 μM CM-H2DCFDA, incubated for 45 min at 37° C., and analyzed with an HTS 7000 Bio Assay Fluorescent Plate Reader (Perkin Elmer) using the HTSoft program.

Animals. 8 week old GLP-1 receptor knockout mice on the C57B16 background were used (Hansotia et al., 2007). All procedures were performed in accordance with the Guide for Care and Use of Laboratory Animals of the National Institutes of Health and were approved by the Animal Subjects Committee of the Albert Einstein College of Medicine. Animals were made diabetic by five daily injections of 50 mg/kg of streptozotocin after an eight hr fast. The animals were test-bled from the tail two weeks after the initial injection. The diabetic animals were randomized into two groups according to their blood glucose levels. Only mice with glucose levels greater than 300 mg/100 ml were used. An ALZET osmotic pump containing 100 μg/ml of GLP-1 (9-36 NH₂) was surgically placed under the skin of a group of diabetic GLP-1 receptor knockout mice. The pump delivered GLP-1 (9-36 NH₂) at a rate of 0.5 μl/hr for 7 days. A bolus injection of 2.5 μg was given at the time of the surgery. A second group of diabetic GLP-1 receptor knockout mice received no treatment and a third group of GLP-1 receptor knockout mice served as non diabetic controls. After seven days the mice were anaesthetized and the aorta removed and used for prostacyclin synthase and eNOS activity assays.

Prostacyclin synthase activity. Activity in cell lysates was determined by measuring levels of 6-keto PGF-1α, a stable product which is produced by the nonenzymatic hydration of PGI₂. A competitive immunoassay method (Correlate-EIA) was used for the quantitative determination of 6-keto-PGF-1α, according to the manufacturer's instructions (Assay Design Inc). For 6-keto-PGF-1α determination in mouse aortas, the aortas were dissected from the abdominal bifurcation to the aortic arch. The aortas were washed with PBS and incubated at 37° C. for three hours in 400 μL incubation buffer.

Determination of 3-nitrotyrosine-modified prostacyclin synthase. Aortas were homogenized in 1 ml cold lysis buffer (50 mM Tris-HCl (pH 7.6), 1% NP-40, 0.25% Na-deoxycholate, 150 mM NaCl, 1 mM EDTA, 1 mM PMSF, Aprotinin, Leupeptin, Pepstatin, 1 mM Na₃VO₄, 1 mM NaF), and incubated at 4° C. with rotation for 60 min. Samples were pelleted for 20 min a 20,000×g at 4° C. 500 μg protein was immunoprecipitated with 4 μg of 3-nitrotyrosine antibody and 20 μl of Protein A-agarose in PBS. Samples were rotated overnight at 4° C., and the IP complexes were pelleted by centrifugation (10,000×g) and washed 4 to 5 times with PBS. The pellet was resuspended in 1× sample buffer, boiled, and analyzed by 7.5% SDS polyacrylamide gel electrophoresis (PAGE) with western-blotting for PGI₂.

eNOS activity. eNOS activity in cell lysates was determined as previously described (Du et al., 2001). Six hours before the determination, media without arginine was added to the cells. Activity of eNOS in cell lysates and tissue was also determined by a previously described immunoprecipitation assay (Garcia-Gardena et al., 1996). Samples were split into two tubes, one for determination of eNOS activity one for Western blotting.eNOS activity was determined by measuring the conversion of [³H]L-arginine into [³H]L-citrulline. All enzyme activities were corrected for [³H]L-arginine uptake into the cells under the various experimental conditions, as previously described (Massillon et al., 1997). eNOS immunocomplexes immobilized on protein A-Sepharose beads were resuspended in assay buffer, run on SDS-PAGE gels, and quantitated by immunoblotting. eNOS activity in aortas from mice was determined using 3 mouse aortas/sample. After immunoprecipitation from tissue lysate, eNOS activity was determined by incubation with 100 μl of reaction buffer (3 μM tetrahydrobiopterin, 1 mM NADPH, 2.5 mM CaCl₂, 200U calmodulin, and ³H-L-arginine (0.2 μCi) for 45 min at 37° C. with rolling. After the incubation, samples were loaded on the Tris-form of DOWEX 50WX8 ion-exchange columns and ³H-citrulline collected. ³H-Citrulline was detected using a scintillation counter.

Immunoprecipitation and Western blotting. Immunoprecipitated proteins electrophoresed on 10% PAGE gels were transferred onto nitrocellulose membranes. The immunoblots were developed with 1:1000 dilutions of primary antibody and anti-RABBIT IRDye™ 800CW (green) and anti-MOUSE (or goat) ALEXA680 (red). Membranes were scanned and quantitated by the ODYSSEY Infrared Imaging System (LI-COR, NE).

Statistics. Data were analyzed using one-factor ANOVA to compare the means of all the groups. The Tukey-Kramer multiple comparisons procedure was used to determine which pairs of means were different.

### Results

GLP-1 Degradation Product Prevents Nutrient-Induced Reactive Oxygen Species in Endothelial Cells. The time-course of increased ROS production was determined in response to concentrations of glucose and free fatty acids found in people with obesity and diabetes (12 mM glucose and 800 μM oleic acid with 1 mM albumin). In response to high glucose (HG), ROS increased 2.7-fold compared to 5 mM glucose (LG) by 30 minutes, and 3.3-fold at 90 minutes. In response to oleic acid, ROS increased 2.4-fold by 90 minutes, and 3.5-fold by 360 minutes. Based on these data, the effect of the inactive metabolites of GLP-1 on HG-induced ROS was assessed at 2 hrs and the effect on oleic acid-induced ROS at 360 min. High glucose increased ROS levels 2.7-fold compared to 5 mM glucose. The presence of either bioactive form of GLP-1 prevented this increase. However, when inhibitors were included of the two enzymes responsible for GLP-1 degradation (DPP-4 and neutral endopeptidase 24.11), bioactive GLP-1 had no effect on HG-induced ROS. In contrast, addition of the “inactive” GLP-1 degradation product in the presence of protease inhibitors completely inhibited high glucose-induced overproduction of ROS. Furthermore, blockade of the GLP-1 receptor with the GLP-1R antagonist exendin 9-39^{amide} had no effect on the ability of the GLP-1 degradation products to prevent glucose-induced overproduction of ROS, suggesting that these peptides do not signal through the known GLP-1 receptor (Thorens 1992). Consistent with this hypothesis, 100-fold excess of bioactive GLP-1 did not affect the ability of GLP-1 [9-36^{amide}] to prevent nutrient-induced ROS. Similar results were observed with fatty acid-induced ROS.

GLP-1 Degradation Product Prevents Inhibition of Anti-Atherogenic Enzyme Activity by Nutrient-Induced ROS. The effect of GLP-1 [9-36^{amide}] was evaluated on nutrient-induced inactivation of two important anti-atherogenic enzymes: prostacyclin (PGI₂) synthase (de Leval et al., 2004; Zou et al., 2002a) and eNOS (Kuhlencordt et al., 2001; Zou et al., 2002b). The critical role of both in atherogenesis has been demonstrated using gene knockout models (Kobayashi et al., 2004; Kuhlencordt et al., 2001). High glucose reduced PGI₂ synthase activity in arterial endothelial cells by 95% compared to 5 mM glucose. GLP-1 [9-36^{amide}] prevented this decrease. Oleic acid reduced PGI₂ synthase activity in these cells to a similar degree, and GLP-1 [9-36^{amide}] also prevented this decrease. eNOS activity was decreased to 25.6% of control by both high glucose and by oleic acid, and GLP-1 [9-36^{amide}] also prevented these decreases.

GLP-1 Degradation Product Reverses Diabetes-Induced Defects in Aortas of GLP-1 Receptor −/− Mice. GLP-1 [9-36^{amide}] did not seem to signal through the known GLP-1 receptor because blockade of the GLP-1 receptor with the GLP-1R antagonist exendin 9-39^{amide} (Goke et al., 1996) had no effect on the ability of the GLP-1 degradation products to prevent glucose-induced overproduction of ROS in cultured cells. However, it is possible that exendin 9-39^{amide} did not fully block the GLP-1 receptor present. To resolve this question using a complementary genetic approach, the effects of GLP-1 [9-36^{amide}] were evaluated on aortic endothelial cell PGI₂ synthase and eNOS activity in diabetic GLP-1 receptor homozygous knockout (Glp1r−/−) mice (Scrocchi et al., 1996). Diabetes reduced PGI₂ synthase activity in aortas of Glp1r−/− mice by 95%, similar to the effect of high glucose in cultured human aortic endothelial cells. Administration of GLP-1 [9-36^{amide}] reversed this established abnormality in diabetic mice, and restored activity to levels not statistically different from cells obtained from non-diabetic mice. Since nutrients inactivate PGI₂ synthase in endothelial cells by reactive oxygen-mediated tyrosine nitration (de Leval et al., 2004; Zou et al., 2002a), the effects of GLP-1 [9-36^{amide}] on PGI₂ synthase tyrosine nitration were also examined. Diabetes increased PGI₂ synthase tyrosine nitration by 2.8-fold. GLP-1 [9-36^{amide}] reversed this established abnormality in diabetic mice, and restored PGI₂ synthase tyrosine nitration to non-diabetic levels. Finally, the effects of GLP-1 [9-36^{amide}] were examined on eNOS activity. Increased ROS inhibits eNOS activity by PKC activation (Naruse et al., 2006), hexosamine pathway activation (Du et al., 2001) and oxidative uncoupling of the eNOS dimmer (Zuo et al. 2002a). Diabetes decreased eNOS activity by 81%. GLP-1 [9-36^{amide}] reversed this established abnormality in diabetic mice, and restored eNOS activity to levels not statistically different from non-diabetic values. Together, these data demonstrate that GLP-1 [9-36^{amide}] does not exert its actions through the GLP-1 receptor. Equally important from the therapeutic point of view, these data also demonstrate that GLP-1 [9-36^{amide}] reverses, as well as prevents, nutrient-induced increases in endothelial ROS production and their consequent inactivation of PGI₂ synthase and eNOS.

### Discussion

These results demonstrate that GLP-1 [9-36^{amide}], commonly assumed to be an inactive product generated by DPP-4-mediated cleavage of GLP-1 in vivo, has a surprising biologic activity both in cultured human aortic endothelial cells and in diabetic mice: prevention of excess mitochondrial ROS production and its deleterious consequences caused by increased glucose and fatty acid levels. This activity is not mediated through interaction with the GLP-1 receptor. The present results indicate that GLP-1 [9-36^{amide}] and GLP-1 [9-37] have beneficial effects for prevention and treatment of vascular dysfunction in people with obesity and diabetes.

### EXAMPLE 6

### GLP-1 (9-36) Confers Protection Against Acute Myocardial Ischemia-Reperfusion Injury in Diabetes Mellitus

Cardiovascular disease is the leading cause of diabetes related death. GLP-1(9-36) inhibits hyperglycemia-induced production of oxidant species in cultured vascular endothelial cells and prevents the inactivation of both eNOS and prostacyclin synthase. The cardioprotective effects of GLP-1(9-36) was investigated in two in vivo diabetic murine models of myocardial ischemia-reperfusion (MI-R) injury.

### Methods

Diabetic (db/db (Type 2 diabetes model) and STZ-diabetic (Type 1 diabetes model)) mice were treated with 2.4 μg/day of GLP-1(9-36) via Alzet pump for 7 days and subjected to 45 min of left coronary artery occlusion and 2 hr of reperfusion. At 2 hr of reperfusion, hearts were excised and evaluated for infarct (INF) size and area-at-risk (AAR) using Evan's blue and 2,3,5-triphenyltetrazolium chloride (TTC) staining. The area at risk is the area supplied by the coronary artery to be occluded.

### Results and Discussion

Both models of diabetic mice exhibited elevated baseline blood glucose values of 386±25 and 476±35 mg/dl respectively. After 7 days of GLP-1(9-36) therapy, db/db mice exhibited a 48% reduction in blood glucose (BG) values. No significant reduction in BG was observed in the STZ-diabetic mice. Importantly, GLP-1 (9-36) reduces myocardial infarct size in diabetic mice. Diabetic (db/db) and STZ-diabetic mice treated with GLP-1(9-36) exhibited a 37% and 45% reduction, respectively, in the percentage of the area-at-risk that was infracted after coronary artery occlusion. GLP-1(9-36) therapy significantly reduced the percentage of the left ventricle (p=0.0002) that was infarcted after the coronary artery occlusion, as studied in STZ-treated mice.

Serum levels of Serum levels of troponin I were also measured in STZ-treated mice. Troponin I is a cardiac muscle-specific protein, which is released by dead cardiac cells. The blood level of troponin I is a proxy for the extent of myocardial cell death. GLP-1(9-36) therapy significantly reduced (p=0.04) serum levels of troponin I, which is indicative of reduced myocardial cell death.

These results indicate that administration of GLP-1(9-36) confers cardioprotection in diabetic mice by attenuating the extent of myocardial injury and cell death following ischemia-reperfusion. Furthermore, GLP-1(9-36) mediated cardioprotection appears to be independent of its anti-hyperglycemic effects.

### EXAMPLE 7

### GLP-1 (9-36) Lowers Blood Glucose in Type 2 Diabetic Mice but not in Type 1 Diabetic Mice

Fasting blood glucose levels were measured in db/db mice, a model of Type 2 diabetes, before and after 5 days treatment with GLP-1 (9-36) via subcutaneous Alzet osmotic pump or vehicle. GLP-1 (9-36) significantly lowered blood glucose in these Type 2 diabetic mice. In contrast, in mice with Type 1 diabetes (induced by the chemical streptozotocin), treatment with GLP-1 (9-36) had no effect on blood glucose. The deduction from these two data sets is that the peptide itself cannot act like insulin (Type 1 diabetics have no insulin secreting cells and virtually no circulating insulin). In contrast, Type 2 diabetics have normal to high levels of circulating insulin, but the tissues on which it works are insensitive (resistant) to its actions. A major insulin target tissue in Type 2 diabetes is the liver, because it can produce huge amounts of glucose and release this into the blood, by a process called gluconeogenesis; this process is inhibited by insulin. Excessive gluconeogenesis is a major cause of hyperglycemia in people with diabetes, and can elevate blood glucose even in the absence of food intake.

### EXAMPLE 8

### GLP-1 (9-36) by Itself has no Effect on Gluconeogenesis, but in the Presence of Insulin, GLP-1 (9-36) Augments the Inhibitory Effect of Insulin on Gluconeogenesis

Drugs that augment the effect of insulin are called insulin sensitizers. Currently, there has been much controversy over potential serious side effects of the thiazolidinedione class of insulin sensitizer drugs, Avandia and Actos. Hence there is a need for insulin sensitizers with reduced side effects. These findings have important clinical implications. Many non-diabetic patients develop acute, stress-induced hyperglycemia during acute medical events such as critical care illness, acute myocardial infarction, and stroke. In intensive care unit patients, titrating insulin infusion to maintain blood glucose levels below 110 mg/dl strikingly reduced mortality by 50% when compared with those whose blood glucose levels were maintained at 150-160 mg/dl. Acute hyperglycemia predicted a 3.8-fold increased risk of in-hospital mortality after ischemic stroke in non-diabetic patients, and a 1.4-fold increased risk from poor functional recovery in nondiabetic stroke survivors. However, a serious problem with exogenous insulin treatment is insulin-induced hypoglycemia. In contrast, treatment with an insulin-sensitizer would avoid hypoglycemia, since the patient's insulin-secreting cells sense blood glucose levels and reduce their insulin secretion to maintain blood glucose in the normal range.

### Methods

Liver cells were isolated from non-diabetic control strain rats and from SDF rats. Single-cell suspensions of hepatocytes were obtained from perfusions using the procedure of Berry and Friend (1969) and the perfusion mixture of Leffert et al. (1979). The cells were plated on tissue culture plastic for 6 hours at a density of 2×10⁵ cells per well in a 24-well culture plate that was pre-coated with rat-tail collagen I. During plating, cells were cultured in RPMI 1640 medium supplemented with 10% FBS, penicillin/streptomycin, 10 μg/ml insulin and 10 μM dexamethasone. After allowing for adherence, the media was changed to RPMI with 5 mM glucose, 0.4% FCS, and no insulin or dexamethasone. The cells were allowed to equilibrate overnight in this low-glucose media. The following morning this media was refreshed, insulin alone, or insulin and GLP-1(9-36) were added and treatment lasted another 24 hours. After stimulation, glucose production was measured by incubating the cells for 6 hours in glucose-free RPMI containing 5 mM each of alanine, valine, glycine, pyruvate and lactate. Glucose was subsequently measured with a Trinder assay (Sigma). Averages were obtained of 4 independent measurements.

In non-diabetic control strain rats, 1000 pM insulin (without GLP-1(9-36)) reduced gluconeogenesis to 46% of control levels. In contrast, in the presence of GLP-1(9-36), only 70 pM insulin was required to reduce gluconeogenesis to 33% of control levels. Similarly, with SDF rats, at an insulin concentration of 70 picomolar, the effect with GLP-1(9-36) on inhibiting gluconeogensis by the liver cells is maximal, and less than even 1000 pM insulin alone. There is no difference between the “insulin only” values of the two different rat cells, suggesting that the insulin resistance in the whole animal is not an intrinsic property of the liver cells, but rather, is due to abnormal signals from other organs (e.g., brain, adipose and/or muscle).

In view of the above, it will be seen that the several advantages of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification are hereby incorporated by reference. The discussion of the references herein is intended merely to summarize the assertions made by the authors and no admission is made that any reference constitutes prior art. Applicants reserve the right to challenge the accuracy and pertinence of the cited references.

APPENDIX
SEQ ID Nos

SEQ ID NO: 1

GLP-1 (9-36)
Glu Gly Thr Phe Thr Ser Asp Val Ser Ser Tyr Leu
Glu Gly Gln Ala Ala Lys Glu Phe Ile Ala Trp Leu
Val Lys Gly Arg

SEQ ID NO:2

GLP-1 (9-37)
Glu Gly Thr Phe Thr Ser Asp Val Ser Ser Tyr Leu
Glu Gly Gln Ala Ala Lys Glu Phe Ile Ala Trp Leu
Val Lys Gly Arg Gly

SEQ ID NO:3

GLP-1 (9-36 + arg37)
Glu Gly Thr Phe Thr Ser Asp Val Ser Ser Tyr Leu
Glu Gly Gln Ala Ala Lys Glu Phe Ile Ala Trp Leu
Val Lys Gly Arg Arg

SEQ ID NO:4

GLP-1 (9-36) acyl-Lys26
Glu Gly Thr Phe Thr Ser Asp Val Ser Ser Tyr Leu
Glu Gly Gln Ala Ala acLys Glu Phe Ile Ala Trp Leu
Val Lys Gly Arg

SEQ ID NO:5

GLP-1 (9-37) acyl-Lys26
Glu Gly Thr Phe Thr Ser Asp Val Ser Ser Tyr Leu
Glu Gly Gln Ala Ala acLys Glu Phe Ile Ala Trp Leu
Val Lys Gly Arg Gly

SEQ ID NO:6

GLP-1 (9-36) acyl-Lys26 + arg 37
Glu Gly Thr Phe Thr Ser Asp Val Ser Ser Tyr Leu
Glu Gly Gln Ala Ala acLys Glu Phe Ile Ala Trp Leu
Val Lys Gly Arg Arg

SEQ ID NO:7

GLP-1 (9-36) acyl-Lys34
Glu Gly Thr Phe Thr Ser Asp Val Ser Ser Tyr Leu
Glu Gly Gln Ala Ala Lys Glu Phe Ile Ala Trp Leu
Val acLys Gly Arg

SEQ ID NO:8

GLP-1 (9-37) acyl-Lys34
Glu Gly Thr Phe Thr Ser Asp Val Ser Ser Tyr Leu
Glu Gly Gln Ala Ala Lys Glu Phe Ile Ala Trp Leu
Val acLys Gly Arg Gly

SEQ ID NO:9

GLP-1 (9-36) acyl-Lys34 + arg 37
Glu Gly Thr Phe Thr Ser Asp Val Ser Ser Tyr Leu
Glu Gly Gln Ala Ala Lys Glu Phe Ile Ala Trp Leu
Val acLys Gly Arg Arg

SEQ ID NO:10

GLP-1 (9-36) acyl-Lys34 and acyl-Lys26
Glu Gly Thr Phe Thr Ser Asp Val Ser Ser Tyr Leu
Glu Gly Gln Ala Ala acLys Glu Phe Ile Ala Trp Leu
Val acLys Gly Arg

SEQ ID NO:11

GLP-1 (9-36) acyl-Lys34 and acyl-Lys26 + arg37
Glu Gly Thr Phe Thr Ser Asp Val Ser Ser Tyr Leu
Glu Gly Gln Ala Ala acLys Glu Phe Ile Ala Trp Leu
Val acLys Gly Arg Arg

SEQ ID NO:12

GLP-1 (9-37) acyl-Lys34 and acyl-Lys26
Glu Gly Thr Phe Thr Ser Asp Val Ser Ser Tyr Leu
Glu Gly Gln Ala Ala acLys Glu Phe Ile Ala Trp Leu
Val acLys Gly Arg Gly

SEQ ID NO:13

GLP-1 (9-37) + Arg38
Glu Gly Thr Phe Thr Ser Asp Val Ser Ser Tyr Leu
Glu Gly Gln Ala Ala Lys Glu Phe lIe Ala Trp Leu
Val Lys Gly Arg Gly Arg

SEQ ID NO:14

GLP-1 (9-37) acyl-Lys34 + Arg38
Glu Gly Thr Phe Thr Ser Asp Val Ser Ser Tyr Leu
Glu Gly Gln Ala Ala Lys Glu Phe Ile Ala Trp Leu
Val acLys Gly Arg Gly Arg

SEQ ID NO:15

GLP-1 (9-37) acyl-Lys26 + Arg38
Glu Gly Thr Phe Thr Ser Asp Val Ser Ser Tyr Leu
Glu Gly Gln Ala Ala acLys Glu Phe lIe Ala Trp Leu
Val Lys Gly Arg Gly Arg

SEQ ID NO:16

GLP-1 (9-37) acyl-Lys34 and acyl-Lys26 + Arg38
Glu Gly Thr Phe Tbr Ser Asp Val Ser Ser Tyr Leu
Glu Gly Gln Ala Ala acLys Glu Phe Ile Ala Trp Leu
Val acLys Gly Arg Gly Arg

### REFERENCES

Alessi D R, Caudwell F B, Andjelkovic M, Hemmings B A, & Cohen P (1996) FEBS Lett 399, 333-338.

Berry, M. N. & Friend, B. S. High-yield preparation of isolated rat liver parenchymal cells: a biochemical and fine structure study. J. Cell. Biol. 43, 506-520 (1969)

Brownlee, M. Nature 414: 813-820, 2001.

Buteau, J, Roduit, R, Susini, S, Prentki, M. Diabetologia 42:856-64, 1999.

de Leval X, Hanson J, David J L, Masereel B, Pirotte B, & Dogne J M (2004) Curr Med Chem 11, 1243-1252.

Deacon C F, Johnsen A H, Holst J J. J Clin Endocrinol Metab 80:952-7, 1995a.

Deacon C F, Nauck M A, Toft-Nielsen M, Pridal L, Willms B, Holst J J. Diabetes 44:1126-31, 1995b.

Diabetes Control and Complications Trial Research Group (DCCTRG) N. Engl. J. Med. 329:977-986, 1993.

Drucker, D J. Diabetes 1998 47:159-69. Diabetes 47:159069, 1998.

Du X L, Edelstein D, Dimmeler S, Ju Q, Sui C, & Brownlee M (2001) J Clin Invest 108, 1341-1348.

Edwards C M, Stanley S A, Davis R, Brynes A E, Frost G S, Seal L J, Ghatei M A, Bloom S R. Am J Physiol Endocrinol Metab 281:E155-61, 2001.

Edwards, D., et al., Science 276:1868, 1997.

Garcia-Cardena G, Fan R, Stern D F, Liu J, & Sessa W C (1996) J Biol Chem 271, 27237-27240.

Geraci M W et al., J. Clin. Invest. 103:1509-15, 1999.

Goke R, Fehmann H C, Linn T, Schmidt H, Krause M, Eng J, & Goke B (1993) J Biol Chem 268, 19650-19655.

Hansotia T, Maida A, Flock G, Yamada Y, Tsukiyama K, Seino Y, & Drucker D J (2007) J Clin Invest 117, 143-152.

Hrkach, J. Diabetes 49 (Suppl. 1): 37, 2000.

Hupe-Sodmann K, McGregor G P, Bridenbaugh R, Goke R, Goke B, Thole H, Zimmermann B, Voigt K. Regul Pept. 58:149-56, 1995.

Hupe-Sodmann K, Goke R, Goke B, Thole H H, Zimmermann B, Voigt K, McGregor G P. Peptides 18:625-32, 1997.

Kieffer T J, McIntosh C H, Pederson R A.Endocrinology 136:3585-96, 1995

Kobayashi T, Tahara Y, Matsumoto M, Iguchi M, Sano H, Murayama T, Arai H, Oida H, Yurugi-Kobayashi T, Yamashita J K, et al. (2004) J Clin Invest 114, 784-794.

Kuhlencordt P J, Gyurko R, Han F, Scherrer-Crosbie M, Aretz T H, Hajjar R, Picard M H, & Huang P L (2001) Circulation 104, 448-454.

Leffert, H. L., Koch, K. S., Moran, T. & Williams, M. Liver cells. Methods Enzymol. 58, 536-544 (1979).

Massillon D, Barzilai N, Hawkins M, Prus-Wertheimer D, & Rossetti L (1997) Diabetes 46, 153-157.

Mentlelin, R, Gallwitz, B, Schmidt, W E. Eur J Biochem 214:829-35, 1993.

Naruse K, Rask-Madsen C, Takahara N, Ha S W, Suzuma K, Way K J, Jacobs J R, Clermont A C, Ueki K, Ohshiro Y, et al. (2006) Diabetes 55, 691-698.

Nishikawa, T., Edelstein, D., Du, X. L., Yamagishi, S., Matsumura, T., Kaneda, Y., Yorek, M. A., Beebe, D., Oates, P. J., Hammes, H. P. Nature 404: 787-790, 2000.

Orskov, C, Rabenhoj, L., Wettergren, A, Kofod, H, and Holst, J J. Diabetes 43:535-9, 1994.

Scrocchi L A, Brown T J, MaClusky N, Brubaker P L, Auerbach A B, Joyner A L, & Drucker D J (1996) Nat Med 2, 1254-1258.

Thorens B (1992) Proc Natl Acad Sci USA 89, 8641-8645.

UK Prospective Diabetes Study Group (UKPDSG) Lancet 352: 837-853, 1998.

VanBever, R. et al., Drug Dev. Res. 48:178, 1999.

Van den Berghe et al., New Eng. J. Med. 345:1359, 2001.

Wettergren et al., Peptides 19:877-82, 1998.

Wei, M., Gaskill, S. P., Haffner, S. M. & Stem, M. P. Diabetes Care 21:1167-1172, 1998.

Zou M H, Shi C, & Cohen R A (2002a) Diabetes 51, 198-203.

Zou M H, Shi C, & Cohen R A (2002b) J Clin Invest 109, 817-826.

PCT Patent Application Publication WO 03/061362.

PCT Patent Application Publication WO 02/085406.

U.S. Patent Application Publication 2003/0073626 A1.

U.S. Provisional Patent Application No. 60/474,520.

1. A method of inhibiting hyperglycemia-induced or free fatty acid-induced reactive oxygen formation in a mammalian cell, the method comprising treating the cell with a pharmaceutically acceptable composition comprising GLP-1 (9-36) sufficient to inhibit the hyperglycemia-induced or free fatty acid-induced reactive oxygen formation in the cell.

2-3. (canceled)

4. The method of claim 1, wherein the cell is selected from the group consisting of a nerve cell, a renal mesangial cell, a pancreatic β cell, an adipocyte, a cardiac myocyte, an endothelial cell or a hepatocyte.

5-6. (canceled)

7. The method of claim 1, wherein the cell is in a mammal that has or is at risk for having diabetes, impaired glucose intolerance, stress hyperglycemia, metabolic syndrome, insulin resistance, ischemia/reperfusion injury, endotoxin injury, non-alcoholic steatohepatitis (NASH), alcoholic liver disease, and/or impaired glucose-stimulated insulin secretion.

8-17. (canceled)

18. The method of claim 1, wherein the GLP-1 (9-36) has the sequence of SEQ ID NO:1.

19. The method of claim 1, wherein the GLP-1 (9-36) is an amide.

20. The method of claim 1, wherein the GLP-1 (9-36) further comprises an additional amino acid at the carboxy terminus.

21. The method of claim 20, wherein the additional amino acid is a Gly.

22. The method of claim 20, wherein the additional amino acid is an arginine.

23. The method of claim 1, wherein the GLP-1 (9-36) has the sequence of any one of SEQ ID NOs:2-16.

24-50. (canceled)

51. A method of inhibiting development of disease due to diabetes, impaired glucose tolerance, stress hyperglycemia, metabolic syndrome, insulin resistance, ischemia/reperfusion injury, endotoxin injury, non-alcoholic steatohepatitis (NASH), alcoholic liver disease, and/or impaired glucose-stimulated insulin secretion in a mammal, or conditions resulting therefrom, the method comprising treating the mammal with a pharmaceutically acceptable composition comprising GLP-1 (9-36) sufficient to inhibit development of the disease.

52. The method of claim 51, wherein the disease is an atherosclerotic, microvascular, or neurologic disease.

53. The method of claim 51, wherein the disease is selected from the group consisting of coronary disease, myocardial infarction, atherosclerotic peripheral vascular disease, cerebrovascular disease, stroke, retinopathy, renal disease, neuropathy, and cardiomyopathy.

54. The method of claim 51, wherein the mammal is administered at least one other treatment for inhibiting the effects of diabetes, impaired glucose tolerance, stress hyperglycemia, metabolic syndrome, and/or insulin resistance.

55. A method of reducing hyperglycemia-induced or free fatty acid-induced inactivation of prostacyclin synthase in a mammal, the method comprising treating the mammal with GLP-1 (9-36) sufficient to inhibit the hyperglycemia-induced or free fatty acid-induced reactive oxygen formation in the mammal.

56. The method of claim 55, wherein the mammal has or is at risk for having diabetes, impaired glucose intolerance, stress hyperglycemia, metabolic syndrome, hypoxic pulmonary hypertension, an acute thrombotic event and/or insulin resistance.

57. The method of claim 55, wherein the mammal is at risk for undergoing an acute thrombotic event.

58. The method of claim 57, wherein the acute thrombotic event is a stroke or a heart attack.

59. A method of inhibiting hyperglycemia-induced or free fatty acid-induced decrease in endothelial nitric oxide synthase (eNOS) activity in an endothelial cell in a mammal, the method comprising treating the mammal with GLP-1 (9-36) sufficient to inhibit the hyperglycemia-induced or free fatty acid-induced decrease in eNOS activity in the cell.

60. The method of claim 59, wherein the endothelial cell is part of the vascular tissue of a living mammal.

61. The method of claim 60, wherein the living mammal has or is at risk for having diabetes, impaired glucose intolerance, stress hyperglycemia, metabolic syndrome, hypoxic pulmonary hypertension, an acute thrombotic event and/or insulin resistance.

62. The method of claim 61, wherein the acute thrombotic event is a stroke or a heart attack.

63-66. (canceled)

67. The method of claim 1, wherein the GLP-1 (9-36) sequence comprises at least one acetylated lysine where the acetyl group is a myristoyl group.

68-72. (canceled)
